(19) United States
(12) Patent Application Publication
(10) Pub. No.: US 2017/0071477 A1
(43) Pub. Date: Mar. 16, 2017

(54) SYSTEM FOR DETECTING CORE BODY TEMPERATURE AND METHOD FOR THE SAME

(71) Applicant: NATIONAL CHIAO TUNG UNIVERSITY, HSINCHU (TW)

(72) Inventors: Chin-Teng LIN, HSINCHU (TW); Li-Wei KO, HSINCHU (TW); Dar-Shong LIN, HSINCHU (TW); Bo-Kai LIN, HSINCHU (TW); Tzu-Yu KUO, HSINCHU (TW)

(21) Appl. No.: 15/267,407

(22) Filed: Sep. 16, 2016

(30) Foreign Application Priority Data
Sep. 16, 2015 (TW) 104130642

(51) Int. Cl. A61B 5/01; A61B 5/0404; A61B 5/08; A61B 5/0402; A61B 5/00

(52) U.S. Cl. CPC A61B 5/01; A61B 5/0402; A61B 5/7278; A61B 5/0816; A61B 5/7225; A61B 5/0008; A61B 5/0006; A61B 5/0404; A61B 5/0022

(57) ABSTRACT

A system for detecting a core body temperature includes a detection unit, an ECG wave-filter, a body-temperature detection unit, a processing unit, a breath computing and processing unit, a heart-beat computing and processing unit, and a core body temperature computing and processing unit. The detection unit senses the body, and then the ECG wave-filter and body-temperature detection unit measures the electrical cardiac signal and the shell temperature, respectively. The processing unit collects the signals generated by the ECG wave-filter and body-temperature detection unit and transmits the collected signals to the breath computing and processing unit and the heart-beat computing and processing unit, which generate the core body temperature according to the received signals. Accordingly, it is possible to increase the physical parameters for monitoring the vital signs comprehensively. In addition, a method for detecting a core body temperature is also disclosed.

### CROSS REFERENCE TO RELATED APPLICATIONS

This Non-provisional application claims priority under 35 U.S.C. §119(a) on Patent Application No(s). 104130642 filed in Taiwan, Republic of China on Sep. 16, 2015, the entire contents of which are hereby incorporated by reference.

### BACKGROUND OF THE INVENTION

Field of Invention

The present invention relates to a detection system and, in particular, to a system and method for detecting a core body temperature, which are useful in observation and reminding of the users.

Related Art

There are many methods for detecting the core body temperature. In general, the core body temperature is measured by a thermometer, such as the mercury thermometer, electronic thermometer, ear thermometer, forehead thermometer, or IR thermal imager. However, the measuring method and resolution are varied depending on the different thermometers. In addition, the mercury thermometer and electronic thermometer are contact-type thermometers, so they must contact with the human body for measuring the correct body temperature. The contact portion of the human body is, for example, anus, armpits or mouth for obtaining the rectal temperature, axillary temperature or oral temperature. However, the measurement accuracy of the contact-type thermometers can be easily affected by the environment temperature or the shell temperature.

The contactless-type thermometers, such as the ear thermometer, forehead thermometer, or IR thermal imager, usually have an IR sensor for sensing the shell temperature. The measuring time of the contactless-type thermometer is short. However, the shell temperature varies depending on the changes of the atmosphere and environment, so the measured results are unreliable and some modifications are needed.

If the measured temperature is incorrect or unreliable, it is hard to detect the abnormal physical statuses, which may result in missing the best time for treatment.

Therefore, it is desired to solve the above issues and achieve a better detection accuracy.

### SUMMARY OF THE INVENTION

In view of the foregoing, an objective of the invention is to provide a system and method for detecting a core body temperature, which are useful in observation and reminding of the users.

The present invention combines an ECG wave-filter, a body-temperature detection unit, a processing unit, a breath computing and processing unit, a heart-beat computing and processing unit, and a core body temperature computing and processing unit, which can cooperate to calculate the core body temperature. This configuration can improve the accuracy of the detected core body temperature. When monitoring the physical parameters such as the heart beating, breathing and body temperature, the invention can alert the abnormal statuses immediately.

To achieve the above objective, the present invention discloses a system for detecting a core body temperature, which includes a plurality of detection units, an ECG wave-filter, a body-temperature detection unit, a processing unit, a breath computing and processing unit, a heart-beat computing and processing unit, and a core body temperature computing and processing unit. The detection units contact a body to capture physical signals of the body. The ECG wave-filter connects with at least one of the detection units and is configured for describing an electrical cardiac signal. The body-temperature detection unit connects with at least one of the detection units for measuring a shell temperature of the body. The processing unit collects signals generated by the ECG wave-filter and the body-temperature detection unit. The breath computing and processing unit connects with the processing unit and calculates with the signal generated by the ECG wave-filter to obtain a breath frequency. The heart-beat computing and processing unit connects with the processing unit and calculates with the signal generated by the ECG wave-filter to obtain a heart-beat frequency. The core body temperature computing and processing unit collects the breath frequency, the heart-beat frequency and the shell temperature, and then generates the core body temperature according to the breath frequency, the heart-beat frequency and the shell temperature. In practice, the detection units are attached on a body to capture physical signals of the body, and the ECG wave-filter and the body-temperature detection unit can measure an electrical cardiac signal and a shell temperature of the body, respectively. The processing unit collects the measured data and then transmits the data to the breath computing and processing unit and the heart-beat computing and processing unit for further calculations. Finally, the core body temperature computing and processing unit calculates with the calculated result to obtain the core body temperature.

In one embodiment, the system further includes an AD/DA conversion unit for converting the signals collected by the processing unit, and a transmission module for transmitting the signals.

In one embodiment, the breath computing and processing unit, the heart-beat computing and processing unit and the core body temperature computing and processing unit are configured in a portable device.

In one embodiment, the transmission module is a wired transmission module or a wireless transmission module.

In one embodiment, the breath computing and processing unit calculates with the signal according to an ECG derived respiration (EDR) technology.

In addition, the present invention also discloses a method for detecting a core body temperature. The method includes the following steps of: (a) attaching a plurality of detection units on a body to capture physical signals of the body and using an ECG wave-filter and a body-temperature detection unit to measure an electrical cardiac signal and a shell temperature of the body, respectively; (b) using a processing unit to collect signals generated by the ECG wave-filter and the body-temperature detection unit; (c) using a breath computing and processing unit to receive the signal generated by the ECG wave-filter and to calculate with the received signal to obtain a breath frequency, and using a heart-beat computing and processing unit to receive the signal generated by the ECG wave-filter and to calculate with the received signal to obtain a heart-beat frequency; and (d) using a core body temperature computing and processing unit to receive the breath frequency, the heart-beat frequency and the shell temperature, and to generate the core body temperature according to the breath frequency, the heart-beat frequency and the shell temperature.

In one embodiment, the method further includes a step of: (b1) using an AD/DA conversion unit to receive the signals collected by the processing unit and to convert the received signals.

In one embodiment, the method further includes a step of: (b2) using a transmission module to transmit the signals converted by the AD/DA conversion unit.

In one embodiment, the method further includes a step of: (b3) using a receiving unit to receive the signals transmitted from the transmission module and then to send the received signals to the breath computing and processing unit and the heart-beat computing and processing unit.

In one embodiment, the transmission module is a Bluetooth module.

As mentioned above, the issues of the conventional art that body temperature measured by the conventional thermometers may be inaccuracy, and the abnormal physical statuses may not be detected in time, which can result in missing the best time for treatment, can be overcome by the technology of the present invention.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram showing a system for detecting a core body temperature according to an embodiment of the invention;

FIG. 2 is a block diagram of the system for detecting a core body temperature according to the embodiment of the invention;

FIG. 3 is a schematic diagram showing the implement of the system for detecting a core body temperature according to the embodiment of the invention;

FIG. 4 is a schematic diagram showing the system for detecting a core body temperature applied with a portable device; and

FIG. 5 is a flow chart showing a method for detecting a core body temperature according to an embodiment of the invention.

### DETAILED DESCRIPTION OF THE INVENTION

The present invention will be apparent from the following detailed description, which proceeds with reference to the accompanying drawings, wherein the same references relate to the same elements.

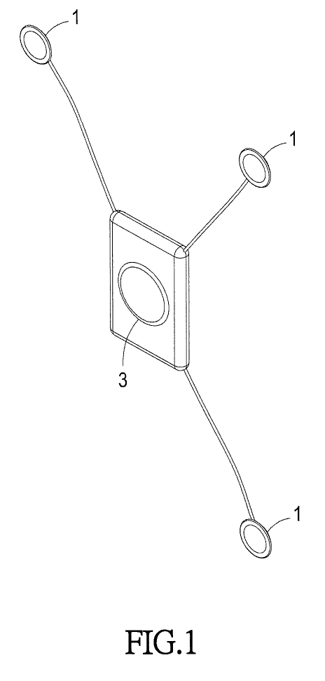

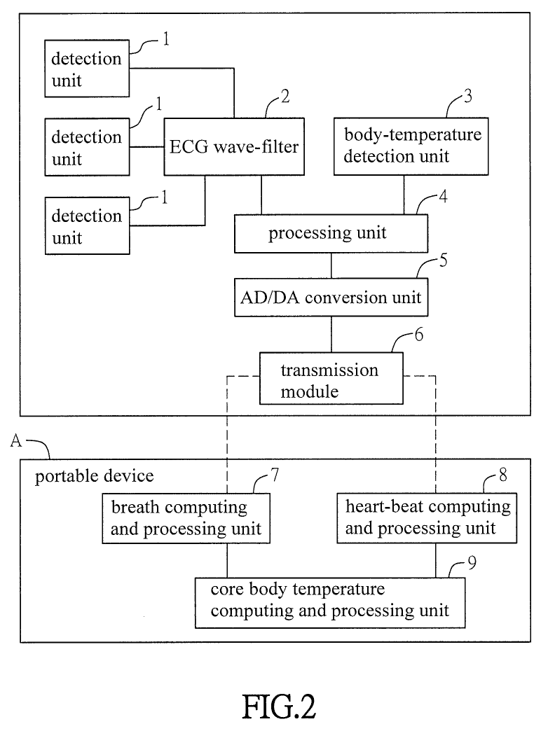

FIG. 1 is a schematic diagram showing a system for detecting a core body temperature according to an embodiment of the invention, and FIG. 2 is a block diagram of the system for detecting a core body temperature. Referring to FIGS. 1 and 2, the system for detecting a core body temperature includes a plurality of detection units 1, an ECG wave-filter 2, a body-temperature detection unit 3, a processing unit 4, an AD/DA conversion unit 5, a transmission unit 6, a breath computing and processing unit 7, a heart-beat computing and processing unit 8, and a core body temperature computing and processing unit 9.

The detection units 1 contact with a body for capturing the physical signals of the body.

The ECG wave-filter 2 is connected with the detection units 1 and is configured for describing an electrical cardiac signal.

The body-temperature detection unit 3 measures a shell temperature of the body.

The processing unit 4 collects the signals generated by the ECG wave-filter 2 and the body-temperature detection unit 3.

The AD/DA conversion unit 5 converts the signals collected by the processing unit 4.

The transmission module 6 is connected with the AD/DA conversion unit 5 and is configured for transmitting the signals. In this embodiment, the transmission module 6 can be a wired transmission module or a wireless transmission module.

The breath computing and processing unit 7 is connected with the processing unit 4 and is configured for calculating with the signal generated by the ECG wave-filter 2 to obtain a breath frequency. In this embodiment, the breath computing and processing unit 7 calculates with the signal according to an ECG derived respiration (EDR) technology.

The heart-beat computing and processing unit 8 is connected with the processing unit 4 and is configured for calculating with the signal generated by the ECG wave-filter 2 to obtain a heart-beat frequency.

The core body temperature computing and processing unit 9 collects the breath frequency, the heart-beat frequency and the shell temperature, which is measured by the body-temperature detection unit 3, and then calculates to generate a core body temperature accordingly.

In addition, the breath computing and processing unit 7, the heart-beat computing and processing unit 8, and the core body temperature computing and processing unit 9 are configured in a portable device A.

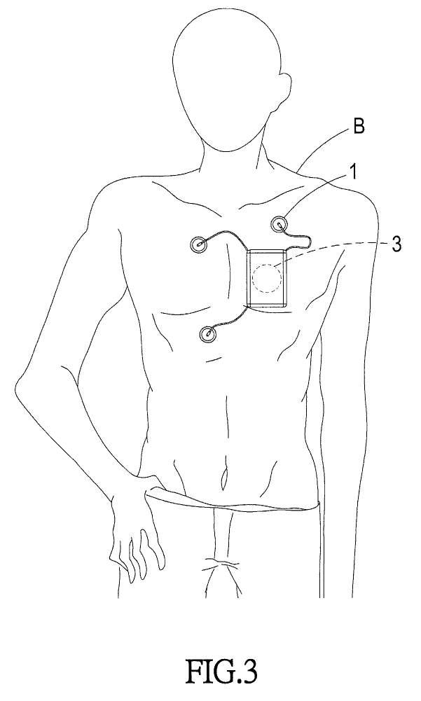

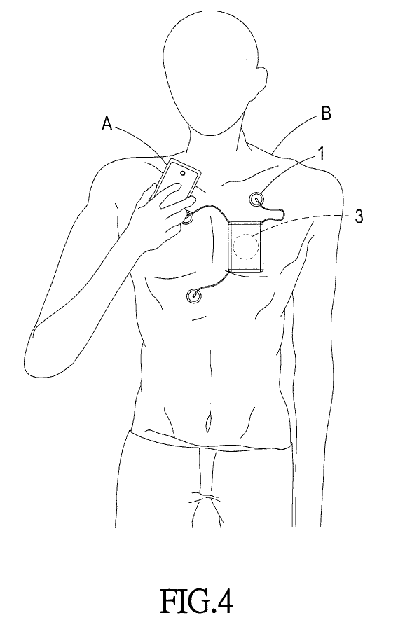

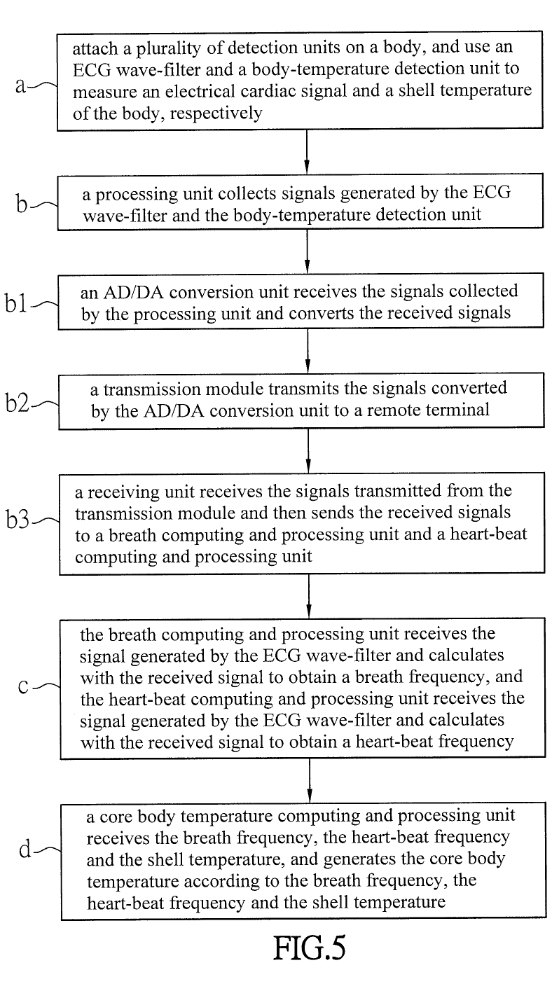

Referring to FIGS. 1 to 5, a method for detecting a core body temperature according to an embodiment of the invention includes the following steps.

A step (a) is to attach a plurality of detection units on a body to capture physical signals of the body, and to use an ECG wave-filter and a body-temperature detection unit to measure an electrical cardiac signal and a shell temperature of the body, respectively.

In a step (b), a processing unit collects signals generated by the ECG wave-filter and the body-temperature detection unit.

In a step (b1), an AD/DA conversion unit receives the signals collected by the processing unit and converts the received signals.

In a step (b2), a transmission module transmits the signals converted by the AD/DA conversion unit to a remote terminal.

In a step (b3), a receiving unit receives the signals transmitted from the transmission module and then sends the received signals to a breath computing and processing unit and a heart-beat computing and processing unit.

In a step (c), the breath computing and processing unit receives the signal generated by the ECG wave-filter and calculates with the received signal to obtain a breath frequency, and the heart-beat computing and processing unit receives the signal generated by the ECG wave-filter and calculates with the received signal to obtain a heart-beat frequency.

In a step (d), a core body temperature computing and processing unit receives the breath frequency, the heart-beat frequency and the shell temperature, and generates the core body temperature according to the breath frequency, the heart-beat frequency and the shell temperature.

The above steps will be described in more detailed in the following example.

At first, three detection units 1 are attached on a body B. The ECG wave-filter 2 is configured for measuring an electrical cardiac signal, and the body-temperature detection unit 3 is configured for measuring a shell temperature of the body B. Next, the electrical cardiac signal and the shell temperature of the body B are transmitted and collected by the processing unit 4. The AD/DA conversion unit 5 receives the signals collected by the processing unit 4 and converts the received signals. After the signal conversion, the converted signals are transmitted to the breath computing and processing unit 7 and the heart-beat computing and processing unit 8 in the portable device A through the transmission module 6 by a wired transmission or a wireless transmission (Bluetooth module). After receiving the converted signals, the breath computing and processing unit 7 calculates with the received signal, which is generated by the ECG wave-filter 2, to obtain a breath frequency according to an ECG derived respiration (EDR) technology. Besides, after receiving the converted signals, the heart-beat computing and processing unit 8 calculates with the received signal, which is generated by the ECG wave-filter 2, to obtain a heart-beat frequency. Finally, the core body temperature computing and processing unit 9 collects the breath frequency, the heart-beat frequency and the shell temperature of the body B, which is measured by the body-temperature detection unit 3, and then generates a core body temperature accordingly.

In summary, the system and method for detecting a core body temperature of the present invention combines an ECG wave-filter, a body-temperature detection unit, a processing unit, a breath computing and processing unit, a heart-beat computing and processing unit, and a core body temperature computing and processing unit, which can cooperate to calculate the core body temperature. This configuration can improve the accuracy of the detected core body temperature. When monitoring the physical parameters such as the heart beating, breathing and body temperature, the invention can alert the abnormal statuses immediately.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A system for detecting a core body temperature, comprising:
a plurality of detection units contacting a body to capture physical signals of the body;
an ECG wave-filter connecting with at least one of the detection units for describing an electrical cardiac signal;
a body-temperature detection unit connecting with at least one of the detection units for measuring a shell temperature of the body;
a processing unit collecting signals generated by the ECG wave-filter and the body-temperature detection unit;
a breath computing and processing unit connecting with the processing unit and calculating with the signal generated by the ECG wave-filter to obtain a breath frequency;
a heart-beat computing and processing unit connecting with the processing unit and calculating with the signal generated by the ECG wave-filter to obtain a heart-beat frequency; and
a core body temperature computing and processing unit collecting the breath frequency, the heart-beat frequency and the shell temperature, and generating the core body temperature according to the breath frequency, the heart-beat frequency and the shell temperature.

2. The system of claim 1, further comprising:
an AD/DA conversion unit for converting the signals collected by the processing unit; and
a transmission module for transmitting the signals.

3. The system of claim 2, wherein the breath computing and processing unit, the heart-beat computing and processing unit and the core body temperature computing and processing unit are configured in a portable device.

4. 4. The system of claim 2, wherein the transmission module is a wired transmission module or a wireless transmission module.

5. The system of claim 1, wherein the breath computing and processing unit calculates with the signal according to an ECG derived respiration (EDR) technology.

6. A method for detecting a core body temperature, comprising steps of:
(a) attaching a plurality of detection units on a body to capture physical signals of the body and using an ECG wave-filter and a body-temperature detection unit to measure an electrical cardiac signal and a shell temperature of the body, respectively;
(b) using a processing unit to collect signals generated by the ECG wave-filter and the body-temperature detection unit;
(c) using a breath computing and processing unit to receive the signal generated by the ECG wave-filter and to calculate with the received signal to obtain a breath frequency, and using a heart-beat computing and processing unit to receive the signal generated by the ECG wave-filter and to calculate with the received signal to obtain a heart-beat frequency; and
(d) using a core body temperature computing and processing unit to receive the breath frequency, the heart-beat frequency and the shell temperature, and to generate the core body temperature according to the breath frequency, the heart-beat frequency and the shell temperature.

7. The method of claim 6, further comprising a step of:
(b1) using an AD/DA conversion unit to receive the signals collected by the processing unit and to convert the received signals.

8. The method of claim 7, further comprising a step of:
(b2) using a transmission module to transmit the signals converted by the AD/DA conversion unit to a remote terminal.

9. The method of claim 8, further comprising a step of:
(b3) using a receiving unit to receive the signals transmitted from the transmission module and then to send the received signals to the breath computing and processing unit and the heart-beat computing and processing unit.

10. The method of claim 8, wherein the transmission module is a Bluetooth module.
